(19) United States
(12) Patent Application Publication
(10) Pub. No.: US 2005/0038307 A1
(43) Pub. Date: Feb. 17, 2005

(54) Zeolite catalysts for transalkylation of diisopropylbenzol

(76) Inventors: Mathieu J. Van der Aalst, Terneuzen (NL); Mohammed S. Samson, Nieuwvliet (NL); Garmt R. Meima, Terneuzen (NL)

Correspondence Address: THE DOW CHEMICAL COMPANY;INTELLECTUAL PROPERTY SECTION, P. O. BOX 1967, MIDLAND, MI, 48641-1967, US

(21) Appl. No.: 10/495,646

(22) PCT Filed: Oct. 7, 2002
(86) PCT No.: PCT/US02/31993

Related U.S. Application Data
(60) Provisional application No. 60/338,352, filed on Dec. 6, 2001.

(51) Int. Cl. C07C 15/12; B01J 29/06

(52) U.S. Cl. 585/475; 502/67; 502/79

(57) ABSTRACT

The present invention disclosed a catalyst composition comprising a dealuminated mordenite component together with a second zeolite which has a 12 membered ring in the acidic form. The catalyst has particular utility in the process for transalkylating dialkylated benzene to form cumene.

### BACKGROUND OF THE INVENTION

The present invention relates to the use of a mixture of two or more zeolites with one being a dealuminated mordenite zeolite catalyst in a transalkylation process to convert di-isopropylbenzene to cumene.

Cumene, also known as isopropylbenzene, is a commercially important compound, for example in the production of phenol and acetone. Cumene is typically prepared by the alkylation of benzene with propylene over zeolite, anhydrous aluminum chloride [AlCl₃] or phosphoric acid catalysts under various conditions. Various processing schemes are known to produce monoalkylaromatic products such as cumene in relatively high yields. However, these existing processes are not without problems such as the production of undesirable by-products. In particular, polyalkylation common in such reactions produces undesirable di- and tri-isopropylbenzene. To reduce formation of these polyalkylates high benzene to propylene ratios can be used in the feed and diluted propylene feedstocks can also be used in some instances.

It is known to react these polyalkylated benzenes with benzene in a separate transalkylation reactor to form cumene, thereby increasing the cumene output. To achieve this, a catalyst is needed which demonstrates a high selectivity to cumene and a high conversion rate of the polyisopropylbenzene.

Various catalysts have been proposed in the art for use in such reactions. These include acidic mordenite zeolites (U.S. Pat. No. 5,243,116), zeolite beta (U.S. Pat. No. 4,891,458, EP 0 687 500), and molecular sieves (EP 0467 007). It is desired to improve upon these catalysts to improve selectivity to cumene and/or conversion of the polyisopropylbenzene.

### SUMMARY AND BACKGROUND OF THE INVENTION

The present invention provides a catalyst suitable for use in a transalkylation step for converting di-isopropyl benzene to cumene. The catalyst comprises a mixture of two or more zeolites with one being a dealuminated mordenite zeolite. The dealuminated mordenite comprises an acidic mordenite zeolite having a silica/alumina molar ratio of at least 30:1. This component of the catalyst should have a crystalline structure which is determined by X-ray diffraction to have a matrix of Cmcm symmetry having dispersed therein domains of Cmmm symmetry. The Symmetry Index is related to the symmetries of the crystals present in the mordenite sample.

The second zeolite component can be any zeolite having a 12 membered ring in the acidic form. The preferred second zeolite component is selected from one or more of Beta zeolite, MCM-22, MCM-36, MCM-49, ERB-1, SSZ-25, Omega and Y zeolite, with Beta Zeolite being most preferred. Zeolite MCM-22 is described in U.S. Pat. No. 4,992,606, Zeolite Y is described in U.S. Pat. No. 3,130,007 and modified forms thereof are described in U.S. Pat. No. 4,459,426 and U.S. Pat. No. 4,798,816. The zeolite Beta component, if present, has the following composition:
[(x/n)M (1+0.1−x)TEA]A10₂·ySiO₂·wH₂O
wherein x is less than 1, y is in the range of 5 to 100, w is in the range of 0 to 4, M is a metal belonging to groups IA, IIA, IIA of the periodic table or is a transition metal, and TEA is tetraethyl ammonium.

Another aspect of the invention is a process for improving any transalkylation reaction in which shape-selective reactions play an important role or reactions in which the formation of a certain isomer is preferred over another, particularly the transalkylation reaction of di-isopropyl-toluene, di-isopropyl-biphenyl or di-isopropyl-naphthalene. The preferred process comprises contacting the benzene and di-isopropyl benzene in the presence of the catalyst under conditions such that cumene is produced. Preferably this process is conducted at the same time as an alkylation reaction of benzene with propylene.

### DETAILED DESCRIPTION OF THE INVENTION

The catalyst of the present invention comprises at least 50, preferably between 60 and 80 percent by weight of a dealuminated mordenite component, and at least 5 to 50, preferably between 20 and 40 percent by weight of a second zeolite which can be any zeolite having a 12 membered ring in the acidic form. The preferred second zeolite component is selected from the group consisting of Beta zeolite, MCM-22, MCM-36, MCM-49, ERB-1, SSZ-25, Omega and Y zeolite (or a mixture of these zeolites).

Zeolite MCM-22 is described in U.S. Pat. No. 4,992,606, Zeolite Y is described in U.S. Pat. No. 3,130,007 and modified forms thereof are described in U.S. Pat. No. 4,459,426 and U.S. Pat. No. 4,798,816. Zeolite beta, including its modified forms, is known in the art as originally described in U.S. Pat. No. 3,308,069 and U.S. Re 28,341 and later described in U.S. Pat. No. 4,891,458 and EP 0 432 814. The zeolite Beta component, if present, has the following composition:
[(x/n)M (1±0.1−x)TEA]AlO₂·ySiO₂·wH₂O

wherein x is less than 1, y is in the range of 5 to 100, w is in the range of 0 to 4, M is a metal belonging to groups IA, IIA, IIA of the periodic table or is a transition metal, and TEA is tetraethyl ammonium.

Optionally the transalkylation catalyst may be bound to, supported on, or extruded with any support material for the purpose of increasing the catalyst's strength and attrition resistance. Suitable supports include aluminas, silicas, aluminosilicates, titania, zirconium, magnesium and clays. Preferably the support is an alumina or silica. The second zeolite catalyst component can be compacted to whatever shape is desired, for example cylindrical extrudates. The second Zeolite component can be produced by any means known in the art, such as those described in EP 0 432 814.

The preferred dealuminated mordenite component of the catalyst of the present invention is also known in the art, see for example U.S. Pat. No. 5,243,116. The preferred dealuminated mordenite component has a silica/alumina molar ratio of at least 30:1, a Symmetry Index (SI), as defined in U.S. Pat. No. 5,243,116 of at least 1.0, and a porosity such that the total pore volume is in the range of from about 0.18 cc/g to about 0.45 cc/g and the ratio of the combined mesopore and macropore volume to the total pore volume is preferably in the range of 0.25 to about 0.75. For purposes of this invention a mesopore has a radius in the range of 3-10 Å and a macropore has a radius in the range of 100-1000 Å. Preferably, the mordenite of the invention has a crystalline structure comprising a matrix of Cmcm symmetry having dispersed therein domains of Cmmm symmetry as those terms are defined in J. D. Sherman and J. M. Bennett, “Framework Structures Related to the Zeolite Mordenite,” Molecular Sieves, J. W. Meier and J. B. Uytterhoeven, eds. Advances in Chemistry Series, 121, 1973, p. 53).

The preferred dealuminated mordenite component can be produced as is described in U.S. Pat. No. 5,243,116.

The transalkylation reaction can be conducted under conditions known in the art, such as those described in U.S. Pat. No. 5,243,116, or EP 0 467007. Preferably the materials are contacted in a continuous flow fixed bed reactor, but other reactor types such as reactive distillation or monolithic reactors may also be used. The second zeolite and dealuminated mordenite catalyst components may be thoroughly mixed or may configured such that the individual components are concentrated in two or more layers.

The reaction conditions are those typically used in the art for such transalkylation reactions. In general the reactor should be at a temperature of from between 120 and 210° C., more preferably between 140 and 180° C. The most preferred temperature will depend on the overall activity of the catalyst mixture and the associated impurity make, in particular the n-propylbenzene formation. The formation of this latter component is undesired and should be controlled at the lowest acceptable level. The pressure should be such that liquid phase reaction conditions are maintained. The feed weight hourly space velocity (WHSV) can be in the range of 0.5 to 50, more preferably 1 to 10, most preferably 1 to5.

### EXAMPLES

The invention will be illustrated by the following Examples. In all of these transalkylation experiments were performed in a continuous flow, fixed bed tubular reactor having an internal diameter of 18.9 mm. The reactor was filled with carborundum (SiC) at the bottom and top with the particular catalyst described in the each example located in between the carborundum layers. The second Zeolite component was a Beta Zeolite (Zeolyst, CP861 DL-25 and had a SiO₂/A1₂O₃ molar ratio of 24:1 and was bound with alumina (20 percent). The dealuminated mordenite had a SiO₂/A1₂O₃ molar ratio of 224:1 and was bound with alumina (20 percent). Liquid phase reaction conditions were maintained with a reaction temperature of 165° C., a pressure of 32 bar and a feed weight hourly space velocity (WHSV) of 1.0 h⁻¹. The feed was an 8:1 mole ratio of benzene to diisopropylbenzene (DIPB). The DIPB had an isomer composition of 40.1 percent by weight meta, 22.4 percent by weight ortho, and 37.5 percent by weight para. Products were analyzed by on-line gas chromatography.

In Example 1 the catalyst bed configuration was mixed, in Example 2 the beta zeolite was located in a layer at the inlet of the reactor and the dealuminated mordenite was located in a layer at the outlet of the reactor, and for Example 3 the dealuminated mordenite was located at the inlet of the reactor and the beta zeolite was at the outlet. The amounts of the catalyst components as well as the results are presented in Table 1.

TABLE 1

| | | | | Con % | Con % | Con | Sel to |
|---|---|---|---|---|---|---|---|
| Example | Beta | | DIPB | meta- | ortho- | para- | Cumene |
| # | Zeolite | Mor | Con % | DIPB | DIPB | DIPB | (mol %) |
| A (comp) | 40 g | 0 | 50 | 14 | 84 | 66 | 89 |
| B (comp) | 0 | 40 g | 52 | 48 | 25 | 72 | 95 |
| 1 | 8 g | 32 g | 62 | 46 | 75 | 71 | 93 |
| 2 | 8 g | 32 g | 60 | 47 | 64 | 71 | 91 |
| 3 | 8 g | 32 g | 62 | 40 | 79 | 75 | 94 |

Con = conversion, Sel = selectivity, Mor = dealuminated mordenite

As can be seen from Table 1, the catalyst composition of the present invention results in higher conversion at equivalent or better selectivity than either component alone.

1) A catalyst composition comprising 50 to 95 percent by weight of a dealuminated mordenite component, and 5 to 50 percent by weight of a second zeolite component which is selected from the group consisting of Beta zeolite, MCM-22, MCM-36, MCM-49, ERB-1, SSZ-25 and Y zeolite.

2) Cancelled.

3) The catalyst composition of claim 1 wherein the second zeolite component is Beta zeolite.

4) The catalyst composition of claim 1 wherein the Beta zeolite component comprises at least 15 percent by weight of the catalyst composition.

5) The catalyst composition of claim 1 wherein the dealuminated mordenite component comprises at least 60 percent by weight of the catalyst composition.

6) The catalyst composition of claim 3 wherein the Beta zeolite component is in the acid form.

7) The catalyst composition of claim 3 wherein the Beta zeolite component is combined with an inorganic binder.

8) The catalyst composition of claim 7 wherein the binder is selected from the group consisting of silicon oxide, aluminum oxide, zirconium oxide, magnesium oxide, natural clays, or mixtures thereof.

9) The catalyst composition of claim 1 wherein the dealuminated mordenite component has a silica/alumina molar ratio of at least 30:1

10) The catalyst composition of claim I wherein the dealuminated mordenite component has a Symmetry Index of at least 1.0.

11) The catalyst composition of claim 1 wherein the dealuminated mordenite component has a porosity such that the total pore volume is in the range of from about 0.18 cc/g to about 0.45 cc/g and the ratio of the combined mesopore and macropore volume to the total pore volume is preferably in the range of 0.25 to about 0.75.

12) The catalyst composition of claim 1 wherein the dealuminated mordenite component is combined with an inorganic binder.

13) A process for transalkylating dialkylated benzene comprising contacting the dialkylated benzene with benzene in the presence of a catalyst under reaction conditions sufficient to form cumene, wherein the catalyst comprises 5 to 50 percent by weight of a zeolite Beta component, and 50 to 95 percent by weight of a dealuminated mordenite component.

14) The process of claim 13 where the dialkylated benzene is contacted with benzene in a continuous flow, fixed bed reactor.

15) The process of claim 14 wherein the feed weight hourly space velocity through the reactor is maintained at a value in the range of 0.5 to 50.

16) The process of claim 13 wherein the catalyst components are mixed together.

17) The process of claim 13 wherein the catalyst components are generally separated into two or more layers.

18) The process of claim 13 wherein the Beta zeolite component comprises at least 15 percent by weight of the catalyst composition and the dealuminated mordenite component comprises at least 60 percent by weight of the catalyst composition.

19) The process of claim 13 wherein the dealuminated mordenite component has a silica/alumina molar ratio of at least 30:1, a Symmetry Index of at least 1.0, and a porosity such that the total pore volume is in the range of from about 0.18 cc/g to about 0.45 cc/g and the ratio of the combined mesopore and macropore volume to the total pore volume is preferably in the range of 0.25 to about 0.75.

20) A process for transalkylating dialkylated benzene to form cumene, the process comprising contacting the dialkylated benzene with benzene in the presence of a catalyst in a continuous flow fixed bed reactor
a) 5 to 50 percent by weight of a zeolite selected from Beta zeolite, MCM-22, MCM-36, MCM-49, ERB-1, SSZ-25 and Y zeolite, wherein the zeolite component further comprises a binder selected from the group consisting of silicon oxide, aluminum oxide, zirconium oxide, magnesium oxide, natural clays, or mixtures thereof; and
b) 50 to 95 percent by weight of a dealuminated mordenite component having a silica/alumina molar ratio of at least 30:1, a Symmetry Index of at least 1.0 and a porosity such that the total pore volume is in the range of from about 0.18 cc/g to about 0.45 cc/g and the ratio of the combined mesopore and macropore volume to the total pore volume is preferably in the range of 0.25 to about 0.75;
